(19) United States
(12) Patent Application Publication
(10) Pub. No.: US 2020/0246497 A1
(43) Pub. Date: Aug. 6, 2020

(54) LUBRICANT FOR MEDICAL DEVICE TO BE SUBJECTED TO GAS LOW-TEMPERATURE STERILIZATION, MEDICAL DEVICE TO BE SUBJECTED TO GAS LOW-TEMPERATURE STERILIZATION, AND METHOD OF MANUFACTURING MEDICAL DEVICE TO BE SUBJECTED TO GAS LOW-TEMPERATURE STERILIZATION

(71) Applicant: OLYMPUS CORPORATION, Tokyo (JP)

(72) Inventors: Minoru HARA, Yokohama-shi (JP); Masaya IWAMOTO, Yamato-shi (JP); Naoyasu HANAMURA, Tokyo (JP); Takashi MAGARA, Tokyo (JP); Koji KOBAYASHI, Tokyo (JP)

(73) Assignee: OLYMPUS CORPORATION, Tokyo (JP)

(21) Appl. No.: 16/855,822

(22) Filed: Apr. 22, 2020

Related U.S. Application Data
(63) Continuation of application No. PCT/JP2018/030641, filed on Aug. 20, 2018.

(30) Foreign Application Priority Data
Oct. 26, 2017 (JP) 2017-207421

(51) Int. Cl. A61L 2/14; A61L 2/26

(52) U.S. Cl. CPC A61L 2/14; A61L 2/26; A61L 2202/15; A61L 2400/10; A61L 2202/24

(57) ABSTRACT

A lubricant for a medical device to be subjected to gas low temperature sterilization includes an anti-friction material and an ion exchanger.

### BACKGROUND

### Field of the Invention

The present invention relates to a lubricant for a medical device to be subjected to gas low-temperature sterilization, the medical device to be subjected to gas low-temperature sterilization, and a method of manufacturing the medical device to be subjected to gas low-temperature sterilization.

This application is a continuation application of a POT International Application No. PCT/JP2018/030641, filed on Aug. 20, 2018, whose priority is claimed on Japanese Patent Application No, 2017-207421, filed in Japan on Oct. 26, 2017. The contents of both the PCT International Application and the Japanese Patent Application are incorporated herein by reference.

### Description of Related Art

In recent years, gas low-temperature sterilization has been widely used as sterilization treatment for a medical device. For example, hydrogen peroxide gas is often used as sterilization gas in the gas low-temperature sterilization.

Examples of a medical device to be subjected to sterilization treatment include devices, such as an endoscope that is used while being inserted into the body, and treatment tools that. are used together with an endoscope. In such a medical device, tubular members or shaft-like members are movably inserted into a flexible tube. A lubricant is used in order to facilitate the movement of the tubular members or the shaft-like members in the flexible tube. The lubricant reduces friction between the inner peripheral surface of the flexible tube and the tubular members or the shaft-like members.

Lubricants for a medical device often include molybdenum disulfide. Molybdenum disulfide is a solid lubricant.

However, sulfur components included in molybdenum disulfide are likely to chemically react with sterilization gas components in a gas low-temperature sterilization process. For example, sulfurous acid, sulfuric acid, and the like are generated in a case where molybdenum disulfide chemically reacts with hydrogen peroxide. As a result, the resin, metal, and the like of the respective members of medical device deteriorate or corrode.

For example, Japanese Unexamined Patent Application, First Publication No. H11-318814 discloses a technique in which a material having a catalytic action on hydrogen peroxide or the low-temperature plasma of hydrogen peroxide is used for a structural member of an insertion unit of an endoscope in order to improve the resistance of the insertion unit of the endoscope to hydrogen. peroxide.

In Japanese Unexamined Patent Application, First Publication No. H11-318814, examples of a material having a catalytic action on the low-temperature plasma of hydrogen peroxide include silver, copper, nickel, palladium, and platinum.

According to the technique disclosed in Japanese Unexamined Patent Application, First Publication No. H11-318814, the amount of hydrogen peroxide acting on a lubricant is reduced to some extents by a catalytic action. Since a reaction process of gas low-temperature sterilization is complex, it is difficult to suppress the chemical reaction of a lubricant by only a catalytic action. Accordingly, a small amount of sulfurous acid, sulfuric acid, and the like are generated due to a chemical reaction at the time of gas low temperature sterilization even though a material having a catalytic action on hydrogen peroxide is added to the structural members of the medical device.

In recent years, the improvement of the cost performance of a medical device has been required in order to reduce medical expenses. It is necessary to improve the resistance of a medical device with respect to gas low-temperature sterilization so as to improve the cost performance of the medical device.

A technique suitable for further reducing the deterioration of a lubricant caused by sterilization gas and the deterioration of structural members of a medical device occurring due to products caused by a chemical reaction between sterilization gas and a lubricant in gas low-temperature sterilization. is desired.

### SUMMARY

According to a first aspect of the present invention, a lubricant for a medical device to be subjected to gas low-temperature sterilization includes an anti-friction material and an ion exchanger.

According to a second aspect of the present invention, in the lubricant for a medical device to be subjected to gas low-temperature sterilization according to the first aspect, the ion exchanger may contain an inorganic substance that can discharge at least one of a hydroxide ion and a proton.

According to a third aspect of the present invention, in the lubricant for a medical device to be subjected to gas low-temperature sterilization according to the first aspect, a content of the ion exchanger may be in a range of 0.1% by mass to 70% by mass.

According to a fourth aspect of the present invention, in the lubricant for a medical device to be subjected to gas low-temperature sterilization according to the first aspect, the ion exchanger and the anti-friction material may be mixed with each other.

According to a fifth aspect of the present invention, in the lubricant for a medical device to be subjected to gas low-temperature sterilization according to the first aspect, the anti-friction material may contain molybdenum disulfide.

According to a sixth aspect of the present invention, a medical device to be subjected to gas low-temperature sterilization includes the lubricant according to the first aspect.

According to a seventh aspect of the present invention, in the medical device to be subjected to gas low-temperature sterilization according to the sixth aspect, the lubricant may be provided in a layer structure on a surface of an adherend.

According to an eighth aspect of the present invention, in the medical device to be subjected to gas low-temperature sterilization according to the seventh aspect, the lubricant may include an anti-friction material layer that includes the anti-friction material as a main component and an ion exchanger layer that includes the ion exchanger as a main component, wherein the anti-friction material layer and the ion exchanger layer may be alternately arranged in a thickness direction thereof.

According to a ninth aspect of the present invention, in the medical device to be subjected to gas low-temperature sterilization. according to the sixth aspect, the medical device may be an endoscope.

According to a tenth aspect of the present invention, in the medical device to be subjected to gas low-temperature sterilization according to the ninth aspect, the endoscope may include a flexible tube and an insertion member that is inserted into the flexible tube, and the lubricant may be provided between an inner peripheral surface of the flexible tube and. an. outer peripheral surface of the insertion member.

According to an eleventh aspect of the present invention, a method of manufacturing a medical device to be subjected to gas low-temperature sterilization includes applying a lubricant including an anti-friction material and an ion exchanger to at least part of a device body of medical device to be subjected to gas low-temperature sterilization.

According to a twelfth aspect of the present invention, in the method of manufacturing a medical device to be subjected to gas low-temperature sterilization according to the eleventh aspect, the process of applying the lubricant may include preparing a material to be applied in which at least the anti-friction material and the ion exchanger are mixed with each other, and applying the material to be applied to the device body.

According to a thirteenth aspect of the present invention, in the method of manufacturing a medical device to be subjected to gas low-temperature sterilization according to the twelfth aspect, the process of applying the lubricant may include forming two or more applied layers including a component of the lubricant on the device body, and the applied layers may be formed by alternately arranging a lubricant layer including the anti-friction material as a main component and an ion exchanger layer including the ion exchanger as a main component.

### BRIEF DESCRIPTION OF THE DRAWINGS

FIG. 1 is a schematic perspective view showing the schematic configuration of an endoscope that is an example of a medical device according to a first embodiment of the invention.

FIG. 2 is a schematic cross-sectional view of an insertion unit of the endoscope that is an example of the medical device according to the first embodiment of the invention.

FIG. 3 is an enlarged view of a portion A of FIG. 2.

FIG. 4 is a schematic cross-sectional view showing an example of the layer structure of a lubricant for a medical device according to a second embodiment of the invention.

FIG. 5A is a diagram showing a step of a method of manufacturing the medical device according to the second embodiment of the invention.

FIG. 5B is a diagram showing a step of the method of manufacturing the medical device according to the second embodiment of the invention.

FIG. 6 is a schematic cross-sectional view showing an example of the layer structure of a lubricant for a medical device of a first modification example according to the second embodiment of the invention.

FIG. 7 is a schematic cross-sectional view showing an example of the layer structure of a lubricant for a medical device of a second modification example according to the second embodiment of the invention.

### DETAILED DESCRIPTION OF EMBODIMENTS

Embodiments of the invention will be described below with reference to the accompanying drawings. The same or corresponding members will be denoted in all drawings by the same reference numerals ever in different embodiments, and description common thereto will be omitted.

### First Embodiment

A lubricant for a medical device to be subjected to gas low-temperature sterilization and a medical device to be subjected to gas low-temperature sterilization according to a first embodiment of the invention will be described below.

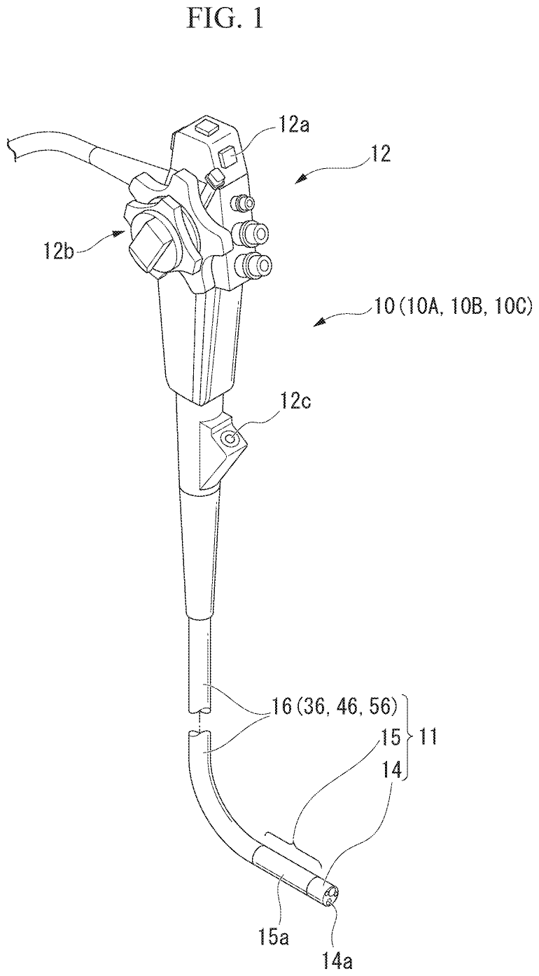

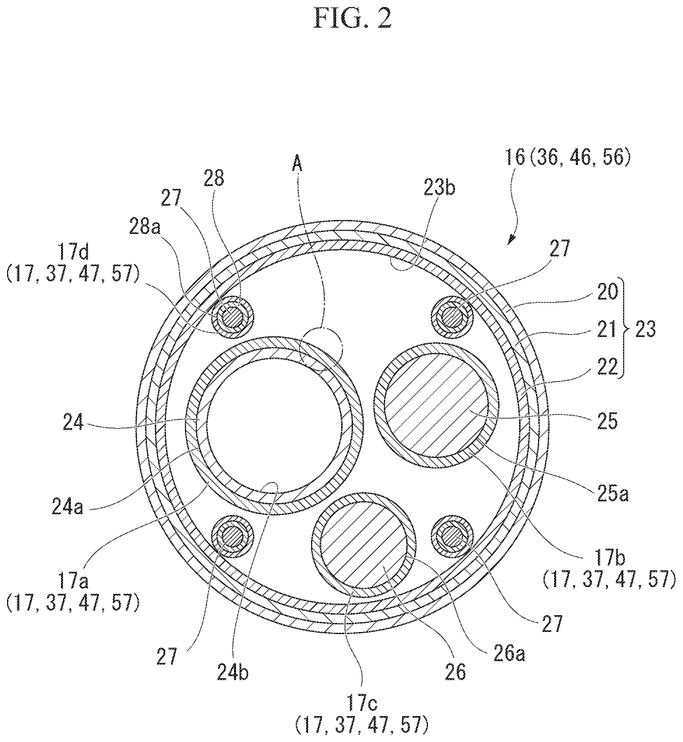

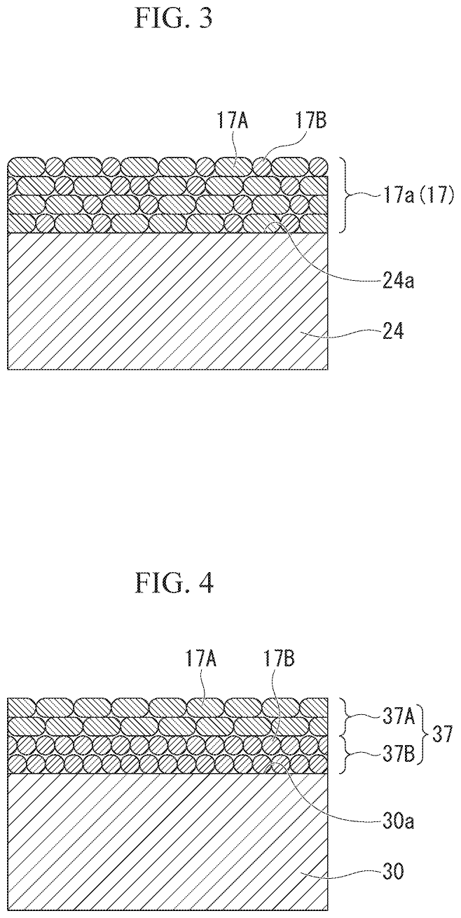

FIG. 1 is a schematic perspective view showing the schematic configuration of an endoscope that is an example of the medical device according to the first embodiment of the invention. FIG. 2 is a schematic cross-sectional view of an insertion unit of the endoscope that is an example of the medical device according to the first embodiment of the invention. FIG. 3 is an enlarged view of a portion A of FIG. 2.

An endoscope 10 (medical device) according to the embodiment shown in FIG. 1 is a medical endoscope that is used while being inserted into the body of a patient. Sterilization treatment to be applied to the endoscope 10 is gas low-temperature sterilization. The type of gas low-temperature sterilization treatment is not particularly limited. Examples of gas low-temperature sterilization treatment suitable for the endoscope 10 include hydrogen peroxide low-temperature plasma sterilization, hydrogen peroxide gas low-temperature sterilization, ethylene oxide gas sterilization, and the like.

The endoscope 10 includes an insertion unit 11 and an operation unit 12.

The insertion unit 11 is formed in the shape of a flexible tube in order to be inserted into the body of a patient. The insertion unit 11 includes a distal end part 14, a bendable part 15, and a flexible tube part 16 that are arranged in this order from the distal end side along an insertion direction. Although not shown in FIG. 1, a treatment tool channel to be described later is provided in the insertion unit 11 in a longitudinal direction so that a treatment tool is inserted into the treatment tool channel.

The distal end part 14 is disposed at a portion that includes the most distal end of the endoscope 10. The distal end part 14 includes an end effector of the endoscope 10 that functions as a manipulator. For example, according to the present embodiment, an image pickup element, such as a CCD, and an image pickup optical system including an appropriate lens are provided in the distal end part 14 in order to acquire the video of an object to be investigated. According to the present embodiment, the distal end part 14 has a columnar shape.

The image pickup element is disposed on the image surface of the image pickup optical system. The image pickup element photoelectrically converts received light to generate image signals.

The image signals generated by the image pickup element are transmitted to the operation unit 12 to be described later through metal wires. The image signals may be subjected to signal processing as necessary before being transmitted to the operation unit 12.

The metal wires include a signal line and a power line. The signal line supplies a control signal to the image pickup element. The power line supplies a drive voltage to the image pickup element. The metal wires are put together in a cable.

However, the image pickup element may be disposed in the operation unit 12 to be described later. In this case, the distal end of an image guide fiber, which transmits a light image to the image pickup element, is disposed on the image surface of the image pickup optical system. The image guide fiber extends up to the operation unit 12, in which the image pickup element is disposed, via the inside of the bendable part 15 and the flexible tube part 16 to be described later. An optical fiber may be used as the image guide fiber.

An image acquired by the distal end part 14 is transmitted as image signals or image light through the metal wires or the optical fiber in the bendable part 15 and the flexible tube part 16, which are to be described later, of the endoscope 10. The metal wires or the optical fiber forms a linear image transmission cable.

The distal end of the distal end part 14 is provided with an image pickup window, an illumination window, and an opening 14a. The opening 14a communicates with a treatment tool channel to be described later.

The bendable part 15 is connected to the proximal end of the distal end part 14. The bendable part 15 is a tubular portion that is adapted to be bendable in order to change the direction of the distal end part 14.

The bendable part 15 includes, for example, a plurality of annular nodal rings. The plurality of nodal rings are rotatably connected to each other. Operation wires to be described later are inserted into the plurality of nodal rings.

For example, linear members, such as electrical wires connected to the image pickup element of the distal end part 14 and a light guide fiber extending up to the illumination window, are housed in the bendable part 15.

The linear members, such as the operation wires, the image transmission cable, and the light guide fiber having been described above, are inserted into the flexible tube part 16 to be described later and extend up to the operation unit 12 to be described later.

The bendable part 15 is covered with a sheath tube 15a.

The flexible tube part 16 is a tubular part that connects the bendable part 15 to the operation unit 12 to be described. later.

As shown in a cross-section in FIG. 2, the flexible tube part 16 includes a flexible tube 23. Long built-in elements, such as a treatment tool channel 24 (insertion member), an image transmission cable 25 (insertion member), a light guide fiber 26 (insertion member), and operation wires 27 are inserted into the flexible tube 23.

The flexible tube 23 includes a flex 22, a SUS blade 21, and a sheath tube 20. The flex 22, the SUS blade 21, and the sheath tube 20 are arranged in this order from the inner peripheral portion of the flexible tube 23 toward the outer peripheral portion thereof.

The flex 22 is formed of a belt-like member that is made of, for example, metal or a resin and is spirally wound. The inner peripheral surface of the flex 22 forms an inner peripheral surface 23b of the flexible tube 23.

The SUS blade 21 is formed in the form of a net-like tube that is woven with a stainless steel wire. The SUS blade 21 covers the flex 22 from the outer peripheral side. The SUS blade 21 overlaps the flex 22.

The sheath tube 20 is a tubular member made of a soft resin. The sheath tube 20 covers the SUS blade 21 from the outer peripheral side. The sheath tube 20 overlaps the SUS blade 21.

According to this structure, the flexible tube 23 can be bent in an appropriate direction in a state where the flexible tube 23 maintains a substantially circular cross-section.

The treatment tool channel 24 is a tubular member that forms a conduit into which an appropriate treatment tool, a catheter, and the like can be inserted. The distal end of the treatment tool channel 24 penetrates the distal end face of the distal end part 14 (see FIG. 1). The distal end of the treatment tool channel 24 forms an opening through which a treatment tool, a catheter, and the like come in and out.

The distal end of the treatment tool channel 24 communicates with the opening 14a (see FIG. 1).

The proximal end of the treatment tool channel 24 is connected to a forceps valve 12c (see FIG. 1) provided on the operation unit 12 to be described later.

The treatment tool channel 24 is formed of a flexible resin tube. The treatment tool channel 24 can be bent together with the flexible tube part 16. It is more preferable that a material allowing a treatment tool, a catheter, and the like being in contact with an inner peripheral surface 24b of the treatment tool channel 24 to easily slide is selected as the resin material of the treatment tool channel 24.

For example, a polyethylene resin, a fluorinated resin, a urethane-based resin, and the like may be used as the material of the treatment tool channel 24.

The image transmission cable 25 transmits an image, which is acquired by the image pickup optical system of the distal end part 14, to the operation unit 12 as image signals or image light. For example, in a case where the image transmission cable 25 transmits image signals, a linear body formed of a metal wire covered with a flexible resin tube is used as the image transmission cable 25. For example, in a case where the image transmission cable 25 transmits image light, a linear body formed of an optical fiber covered with a flexible resin tube is used as the image transmission cable 25.

The light guide fiber 26 supplies illumination light. Illumination light is supplied from the illumination window of the distal end part 14 in order to illuminate the outside. A structure where an optical fiber transmitting illumination light is covered with a flexible resin tube is used as the light guide fiber 26.

The distal end of the light guide fiber 26 is disposed so as to face the illumination window of the distal end part 14. The light guide fiber 26 extends into the flexible tube 23 via the distal end part 14 and the bendable part 15. The proximal end of the light guide fiber 26 is optically coupled to a light source disposed in the operation unit 12 to be described later.

The operation wires 27 transmit a driving force for bending the bendable part 15. For example, in a case where the bendable part 15 is adapted to be bendable in two axis directions, four operation wires 27 are provided as shown in FIG. 2. The distal ends of the respective operation wires 27 are connected to a cap (not shown) provided at the distal end of the bendable part 15. The respective operation wires 27 are separated in diagonal directions orthogonal to each other in the bendable part 15 with the central axis of the bendable part 15 interposed therebetween and are inserted into the nodal rings.

Each operation wire 27 is inserted into a coil sheath 28 (insertion member) for the purpose of maintaining a constant path length in the flexible tube 23 even though the flexible tube 23 is bent. Each coil sheath 28 has a structure where a metal wire is closely wound. Each coil sheath 28 has an inner diameter substantially equal to the outer diameter of the operation wire 27.

The coil sheaths 28 are inserted into the flexible tube part 16. The coil sheath 28 covers the operation wires 27 from the outer peripheral side.

The distal ends of the respective coil sheaths 28 are fixed to a cap (not shown) provided at the proximal end of the bendable part 15. The proximal ends of the respective coil sheaths 28 are fixed to a fixed plate (not shown) provided in the operation unit 12.

Each coil sheath 28 is not particularly fixed in the flexible tube 23. As a result, each coil sheath 28 can be moved in a gap formed in the flexible tube 23. However, the entire length of each coil sheath 28 is not changed even though each coil sheath 28 is moved or bent in the flexible tube 23.

The treatment tool channel 24, the image transmission cable 25, the light guide fiber 26, and the coil sheaths 28 are housed in the flexible tube 23. The treatment tool channel 24, the image transmission cable 25, the light guide fiber 26, and the coil sheaths 28 are parallel to each other in the flexible tube 23. Each of the treatment tool channel 24, the image transmission cable 25, the light guide fiber 26, and the coil sheaths 28 is a flexible linear insertion member.

In a case where the flexible tube 23 is bent, each insertion member is also deformed depending on the deformation of the flexible tube 23. In this case, the respective insertion members slide on each other while being in contact with each other, or slide on the inner peripheral surface 23b of the flexible tube 23 while being in contact with the inner peripheral surface 23b. In this case, a friction force acts between. each insertion member and the flexible tube 23. As a result, a deformation load corresponding to the magnitude of a friction force is generated in a case where the flexible tube 23 is deformed. Since the flexible tube part 16 cannot be smoothly inserted into the body of a patient in a case where a deformation load is increased, a burden on not only an operator but also a patient is also increased.

Accordingly, a lubricant layer 17 (a lubricant, an applied layer) is formed on the surface of each insertion member in this embodiment. In a case where the lubricant layers 17 of the respective insertion members are to be distinguished from each other in the following description, lowercase alphabet letters a, b, c, and d are added for distinguishment. A lubricant layer 17a is a lubricant layer 17 that is formed on an outer peripheral surface 24a of the treatment channel 24. A lubricant layer 17b is a lubricant layer 17 that is formed on an outer peripheral surface 25a of the image transmission cable 25. A lubricant layer 17c is a lubricant layer 17 that is formed on an outer peripheral surface 26a of the light guide fiber 26. A lubricant layer 17d is a lubricant layer 17 that is formed on an outer peripheral surface 28a of each coil sheaths 28.

However, an adherend on which the lubricant layer 17 is to be formed is not limited to the respective insertion members having been described above. As long as the lubricant layer 17 is formed on a member (device body) forming part of the endoscope 10, the member is not particularly limited. For example, the lubricant layer 17 may be provided on the surfaces of appropriate device bodies of the endoscope 10 that slide on each other.

The specific structure of the lubricant layer 17 will be described after the description of the operation unit 12.

As shown in FIG. 1, the operation unit 12 is part of the device that is used by an operator in order to operate the endoscope 10. Examples of an operation using the operation unit 12 can include an operation for pulling the operation wires 27 in order to change the amount of bending of the bendable part 15. The operation unit 12 includes, for example, an operation switch 12a, operation. knobs 12b, and the like.

The forceps valve 12c is provided on the distal end side of the operation unit 12 in order to allow a treatment tool, a catheter, and the like to be inserted into the treatment tool channel 24. The forceps valve 12c includes a valve body that prevents the back flow of fluid in the treatment tool channel 24. As a result, since a treatment tool, a catheter, and the like are inserted through the forceps valve 12c, the treatment tool, the catheter, and the like can be inserted and removed in a state where the back flow of fluid in the treatment tool channel 24 is prevented.

As shown in FIG. 2, the respective lubricant layers 17 are provided in the form of a layer on the outer peripheral surface 24a of the treatment tool channel 24, the outer peripheral surface 25a of the image transmission cable 25, the outer peripheral surface 26a of the light guide fiber 26, and the outer peripheral surface 28a of the coil sheath 28, respectively. The treatment tool channel 24, the image transmission cable 25, the light guide fiber 26, and the coil sheaths 28 form part of the device body of the endoscope 10. The treatment tool channel 24, the image transmission cable 25, the light guide fiber 26, and the coil sheaths 28 are the adherends for the lubricant layers 17.

In this embodiment, the adherends for the lubricant layers 17a, 17b, 17c, and 17d are different from each other but the lubricant layers 17a, 17b, 17c, and 17d have the same structure. The structure of the lubricant layer 17 will be described below using the lubricant layer 17a as an example. The following description of the lubricant layer 17a is also applied to the lubricant layers 17b, 17c, and 17d likewise except for a difference in adherend.

The lubricant layer 17a provided on the outer peripheral surface 24a of the treatment tool channel 24 is schematically shown in FIG. 3.

As schematically shown in FIG. 3, the lubricant layer 17a has a structure where a granular anti-friction material 17A and a granular ion exchanger 175 are provided in the form of a layer on the outer peripheral surface 24a. In this embodiment, the anti-friction material 17A and the ion exchanger 175 are substantially uniformly mixed in the lubricant layer 17a. Appropriate additives, for example, inorganic fillers, organic fillers, and the like may be included in the lubricant layer 17a in addition to the anti-friction material 17A and the ion exchanger 17B.

The thickness of the lubricant layer 17a is not particularly limited as long as a friction reduction effect required for the treatment tool channel 24 is obtained.

The layered structure schematically shown in FIG. 3 is exemplary. The layered structure of the lubricant layer 17a is not limited to the layered structure shown in FIG. 3.

For example, the lubricant layer 17a may have a structure where the anti-friction material 17A and the ion exchanger 17B are multiply stacked in a thickness direction as in the example schematically shown in FIG. 3. In this case, an appropriate thickness may be determined as the thickness of the lubricant layer 17a in consideration of the stability of adhesion of the anti-friction material 17A and the ion exchanger 17B to the outer peripheral surface 24a.

In this embodiment, even though the anti-friction material 17A and the ion exchanger 17B are multiply stacked in the thickness direction, the anti-friction material 17A and the ion exchanger 17B are mixed with each other and dispersed according to the percentage contents thereof as seen in the thickness direction. As a result, both the anti-friction material 17A and the ion exchanger 17B are exposed to the surface of the lubricant layer 17a.

However, the anti-friction material 17A and the ion exchanger 17B of the lubricant layer 17a may be mixed with each other and may be disposed in the state of a single layer as a whole. In this case, the anti-friction material 17A and the ion exchanger 17B are disposed on the outer peripheral surface 24a in a state where the anti-friction material 17A and the ion exchanger 17B are exposed to the outer peripheral surface 24a, It is more preferable that the anti-friction material 17A and the ion exchanger 17B are closely adjacent to each other. However, the anti-friction material 17A and the ion exchanger 17B may be away from each other. The anti-friction material 17A and the ion exchanger 17B may be dispersed in a state where each of the anti-friction material 17A and the ion exchanger 17B is distributed in the shape of an island in a range larger than the particle size thereof.

An appropriate solid lubricant not affecting the durability of an adherend, such as the treatment tool channel 24, is used as the material of the anti-friction material 17A. Examples of the solid lubricant suitable for the anti-friction material 17A include molybdenum disulfide (MoS₂), graphite, fluororesin particles, graphite fluoride, boron nitride, and the like. Examples of the fluororesin particles include polytetrafluoroethylene (PTFE), PFA (a copolymer of tetrafluoroethylene (C₂F₄) and perfluoroalkoxyethylene)), and the like.

The anti-friction material 17A may be formed of one type of solid lubricant, and may be formed of a mixture of a plurality of types of solid lubricants.

The ion exchanger 17B is used in order to improve the sterilization resistance of the anti-friction material 17A or an adherend for the lubricant layer 17.

The inventors have investigated the further improvement of the sterilization resistance of the anti-friction material 17A and an adherend in gas low-temperature sterilization treatment using sterilization gas, in earnest. The inventors have newly found that the sterilization resistance of the anti-friction material 17A and an adherend can be significantly improved in a case where the lubricant layer 17 is formed through combination of the anti-friction material 17A and an ion exchanger used the exchange of ions. As a result, the inventors have reached the invention.

The mechanism of the action of the sterilization gas in the gas low-temperature sterilization is complex. Accordingly, it is not thought that only the presence of ions in the sterilization gas contributes to a chemical reaction related to sterilization in the gas low-temperature sterilization. However, according to the inventors' investigation, in a case where an ion exchanger is included in the lubricant layer 17, better sterilization resistance is obtained as compared to metal particles that are said to have a catalytic action on the sterilizaton gas. Incidentally, the ion exchanger may be called an ion scavenger.

The type of the ion exchanger 17B may be any one of a cation exchanger, an anion exchanger, and an amphoteric ion exchanger. However, it is more preferable that the ion exchanger 17B is an amphoteric ion exchanger.

Examples of a particularly preferred ion exchanger 17B include a composition containing an inorganic substance that can discharge at least one of a hydxoxide ion and a proton.

For example, inorganic compounds including at least one type of metal atoms among bismuth (Bi), antimony (Sb), zirconium (Zr), magnesium (Mg), and aluminum (Al) may be used as the ion exchanger 17B.

Among these inorganic compounds, for example, specific examples of the amphoteric ion exchanger include IXE (registered trademark)-600 (trade name; manufactured by Toagosei Co., Ltd., Sb-Bi-based ion compound), IXE (registered trademark)-633 (trade name; manufactured by Toagosei Co., Ltd., Sb-Si-based ion compound), IXE (registered trademark)-6107 (trade name; manufactured by Toagosei Co., Ltd., Zr-Bi-based ion compound), IXE (registered trademark)-6136 (trade name; manufactured by Toagosei Co., Ltd., Zr-Si-based ion compound), IXEPLAS (registered trademark)-A1 (trade name; manufactured by Toagosei Co., Ltd., Zr-Mg-Al-based ion compound), IXEPLAS (registered trademark)-A2 (trade name; manufactured by Toagosei Co., Ltd., Zr-Mg-Al-based ion compound), IXEPLAS (registered trademark)-B1 (trade name; manufactured by Toagosei Co., Ltd., Zr-Bi-based ion compound), and the like.

For example, specific examples of the anion exchanger include IXE (registered trademark)-700F (trade name; manufactured by Toagosei Co., Ltd., Mg-Al-based ion compound), and the like.

For example, specific examples of the cation exchanger include IXE (registered trademark)-100 (trade name; manufactured by Toagosei Co., Ltd., Zr-based ion compound), and the like.

For example, in a case where hydrogen peroxide gas is used as the sterilization gas and molybdenum disulfide is included as the anti-friction material 17A, IXE (registered trademark)-6107 is particularly suitable as the ion exchanger 17B.

It is more preferable that the percentage content of the ion exchanger 17B in the lubricant layer 17a is in the range of 0.1% by mass to 70% by mass.

There is a concern that it may be difficult for a chemical reaction between the sterilization gas and the anti-friction material 17A to be suppressed in a case where the percentage content of the ion exchanger 17B is less than 0.1% by mass.

There is a concern that the friction reduction performance of the lubricant layer 17a may be reduced due to a relative reduction in the content of the anti-friction material 17A in a case where the percentage content of the ion exchanger 17B exceeds 70% by mass.

Next, a method of forming the lubricant layer 17 of the endoscope 10 will be mainly described with regard to a method of manufacturing the medical device of this embodiment.

In this embodiment, a material to be applied is prepared in order to form the lubricant layer 17. At least the anti-friction material 17A and the ion exchanger 17B are mixed with each other to manufacture the material to be applied. The above-mentioned additives may be contained in the material to be applied in addition to the anti-friction material 17A and the ion exchanger 17B.

Then, the material to be applied is applied to the surface of an adherend. A dry application method or a wet application method is used as a method of applying the material to be applied.

Examples of the dry application method include spray application, rubbing application, and the like. In the case of the rubbing application, for example, a material to be applied may be rubbed on the surface of an adherend while a pressing force is applied to the material to be applied by, for example, an application jig, a hand, or the like. In the case of the rubbing application, for example, a material to be applied adhering to the surface of an adherend may be swept along the surface of the adherend by an application jig, a hand, or the like.

As the wet application method, dispersed liquid to be applied in which a material to be applied is dispersed in a solution to be applied may be formed and may be then applied to an adherend by, for example, spray, dipping, or the like. After that, for example, the solution to be applied is evaporated by the heating of the adherend or the like, so that the lubricant layer 17 is formed on the surface of the adherend.

In this way, the lubricant layers 17a, 17b, 17c, and 17d are formed on the surfaces of the insertion members formed of the treatment tool channel 24, the image transmission cable 25, the light guide fiber 26, and the coil sheaths 28, respectively.

Each insertion member on which the lubricant layer 17 is formed is inserted into the flexible tube 23 as shown in FIG. 2. The insertion members are fixed to fixing counterpart members at fixing positions, respectively. The operation wires 27 are inserted into the coil sheaths 28, respectively.

The endoscope 10 is manufactured as described above.

Next, the action of the lubricant layer 17 will be mainly described with regard to the action of the endoscope 10.

The endoscope 10 is a medical device that is used after being subjected to gas low-temperature sterilization. The endoscope 10 is repeatedly subjected to gas low-temperature sterilization.

In the gas low-temperature sterilization, microorganisms to be subjected to sterilization chemically react with reactive components caused by the sterilization gas and are thus destroyed. However, the reactive components derived from the sterilization gas also chemically attack the structural members of the endoscope 10. As a result, there is a concern that the reactive components caused by the sterilization gas may cause the structural members to deteriorate.

Examples of the reactive components derived from the sterilization gas include ions that are ionized by the sterilization gas, free radicals that are generated due to the sterilization gas, highly reactive intermediates that are generated in a sterilization process, and the like.

According to the lubricant layer 17, since the anti-friction material 17A and the ion. exchanger 17B are mixed with each other, the deterioration of the anti-friction material 17A in a sterilization process is significantly suppressed.

The mechanism of a reaction in a sterilization process is complex. Accordingly, the specific action of the ion exchanger 17B is not specified with regard to an action for suppressing the deterioration of the anti-friction material 17A. However, as the action of the ion exchanger 17B, it is thought that at least ions likely to react with a compound forming the anti-friction material 17A are trapped by the ion exchanger 17B positioned near the anti-friction material 17A.

For example, in a case where molybdenum disulfide is included in the anti-friction material 17A and hydrogen peroxide is used as the sterilization gas, the hydrogen peroxide is chemically combined with sulfur components of the molybdenum disulfide and sulfurous acid and sulfuric acid are generated. In a case where part of the molybdenum disulfide is consumed by a reaction, the lubrication performance of the anti-friction material 17A is reduced due to the destruction of molecular structure having lubricity. In addition, reaction products, such as sulfurous acid and sulfuric acid, cause the structural members of the endoscope 10 to corrode.

The ion exchanger 17B can suppress this chemical reaction of the molybdenum disulfide. As a result, the ion exchanger 17B can prevent a reduction in the lubrication performance of the molybdenum disulfide and the deterioration of the structural members of the endoscope 10 that is caused by reaction products.

According to the lubricant layer 17 of this embodiment and the endoscope 10 including the lubricant layer 17, resistance to gas low-temperature sterilization is improved as described above.

### Second Embodiment

Next, a lubricant for a medical device to be subjected to gas low-temperature sterilization and a medical device to be subjected to gas low-temperature sterilization according to a second embodiment of the invention will be described.

FIG. 4 is a schematic cross-sectional view showing an example of the layer structure of a lubricant for the medical device according to the second embodiment of the invention.

An endoscope 10A (medical device) of this embodiment shown in FIG. 1 is subjected to gas low-temperature sterilization treatment like the endoscope 10 of the first embodiment.

The endoscope 10A includes a flexible tube part 36 instead of the flexible tube part 16 of the endoscope 10 of the first embodiment. As shown in FIG. 2, the flexible tube part 36 includes a lubricant layer 37 (a lubricant, an applied layer) instead of the lubricant layer 17 of the first embodiment.

A difference between the first and second embodiments will be mainly described below.

As schematically shown in FIG. 4, the lubricant layer 37 includes an ion exchanger layer 37B (applied layer) and an anti-friction material layer 37A (applied layer). The ion exchanger layer 37B (applied layer) and the anti-friction material layer 37A (applied layer) are stacked in this order on a surface 30a of an adherend 30.

As long as the adherend 30 is a member forming part of the device body of the endoscope 10A as in the first embodiment, the adherend 30 is not particularly limited. In this embodiment, as in the first embodiment, the adherend 30 corresponds to, for example, the treatment tool channel 24, the image transmission cable 25, the light guide fiber 26, and the coil sheaths 28.

The ion exchanger layer 37B is a layered portion that includes the same ion exchanger 17B as that of the first embodiment as a main component. The ion exchanger layer 37B is formed of one or more layers of the ion exchanger 17B that are stacked in the thickness direction. Appropriate additives and the like may be included in the ion exchanger layer 37B in addition to the ion exchanger 17B.

The anti-friction material layer 37A is a layered portion that includes the same anti-friction material 17A as that of the first embodiment as a main component. The anti-friction material layer 37A is formed of one or more layers of the anti-friction material 17A that are stacked in the thickness direction. Appropriate additives and the like may be included in the anti-friction material layer 37A in addition to the anti-friction material 17A.

It is more preferable that the percentage content of the ion exchanger 17B in the lubricant layer 37 is in the range of 0.1% by mass to 70% by mass as in the first embodiment.

Next, a method of forming the lubricant layer 37 of the endoscope 10A will be mainly described with regard to a method of manufacturing the medical device of this embodiment.

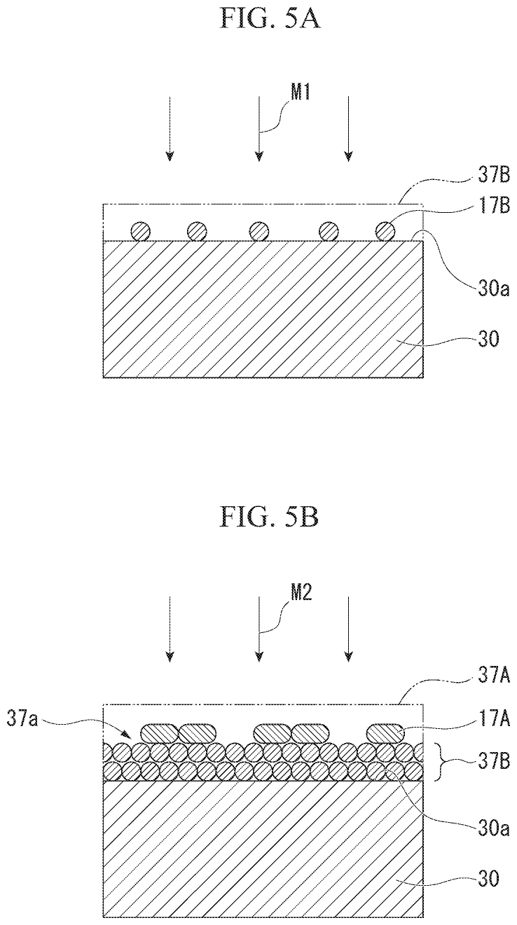

FIGS. 5A and 5B are diagrams showing steps of the method of manufacturing the medical device according to the second embodiment of the invention.

A first material M1 to be applied (see FIG. 5A) that includes the ion exchanger 17B as a main component and a second material M2 to be applied (see FIG. 5B) that includes the anti-friction material 17A as a main component are prepared in this embodiment. The first material M1 to be applied is used in order to form the ion exchanger layer 37F. The second. material M2 to be applied is used in order to form the anti-friction material layer 37A. The above-mentioned additives may be contained in the first material M1 to be applied and the second material M2 to be applied, in addition to the anti-friction material 17A and the ion exchanger 17F, respectively.

Then, the first material M1 to be applied is applied on the surface 30a of the adherend 30 as shown in FIG. 5A. The same application method as the method of applying the material to be applied of the first embodiment is used as a method of applying the first material M1 to be applied.

The first material M1 to be applied is applied to have a predetermined thickness, so that the ion exchanger layer 37B is formed on the surface 30a.

After that, the second material M2 to be applied is applied to a surface 37a of the ion exchanger layer 37B as shown in FIG. 5B. The same application method as the method of applying the material to be applied of the first embodiment is used as a method of applying the second material M2 to be applied.

The second material M2 to be applied is applied to have a predetermined thickness, so that the anti-friction material layer 37A is formed on the surface 37a.

In this way, the lubricant layer 37 is formed on the surface 30a of the adherend 30.

Each insertion member on which the lubricant layer 37 is formed is inserted into the flexible tube 23 as shown in FIG. 2. The insertion members are fixed to fixing counterpart members at fixing positions, respectively. The operation wires 27 are inserted into the coil sheaths 28, respectively.

The endoscope 10A is manufactured as described above.

Next, the action of the lubricant layer 37 will be mainly described with regard to the action of the endoscope 10A.

The ion exchanger layer 37B, which includes the ion exchanger 17B as a main component, of the lubricant layer 37 is disposed between the anti-friction material layer 37A and the adherend 30. As a result, chemical attack on the anti-friction material 17A, which is caused by reactive components permeating the lubricant layer 37 in gas low-temperature sterilization, is suppressed by the same action of the ion exchanger 173 as that of the first embodiment.

As a result, according to the lubricant layer 37 of this embodiment and the endoscope 10A including the lubricant layer 37, resistance to gas low-temperature sterilization is improved.

Further, according to this embodiment, the anti-friction material layer 37A including the anti-friction material 17A as a main component is positioned on the outermost layer of the adherend 30. The friction reduction characteristics of the anti-friction material layer 37A are more excellent than that of the lubricant layer 17 in which the anti-friction material 17A and the ion exchanger 17B are mixed with each other as in the first embodiment. As a result, sliding friction during the use of the endoscope 10A is further reduced.

Furthermore, according to this embodiment, the surface 30a of the adherend 30 is covered with the ion exchanger layer 37B including the ion exchanger 17B as a main component. As a result, the ion exchanger layer 37B also suppresses chemical attack on the adherend 30 that is caused by sterilization gas permeating the anti-friction material layer 37A. Alternatively, the ion exchanger layer 37B suppresses chemical attack on the anti-friction material layer 37A that is caused by sterilization gas permeating the adherend 30.

### FIRST MODIFICATION EXAMPLE

Next, a first modification example of the second embodiment will be described.

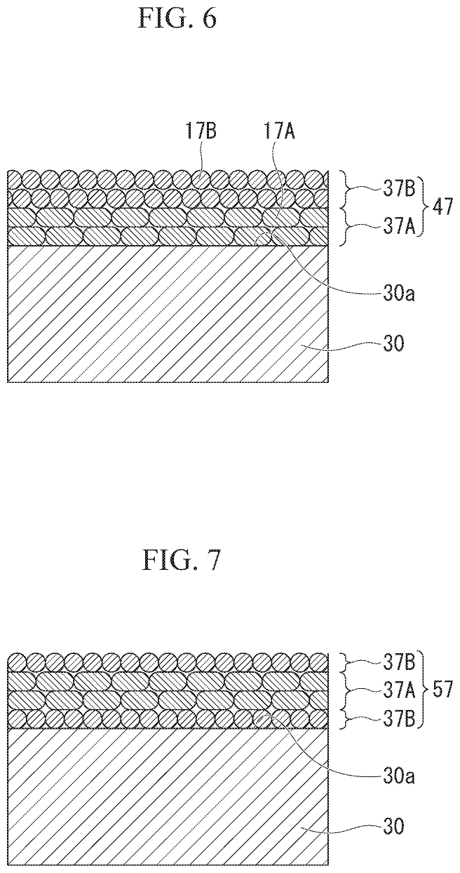

FIG. 6 is a schematic cross-sectional view showing an example of the layer structure of a lubricant for a medical device of a first modification example of the second embodiment of the invention.

An endoscope 10B (medical device) of this modification example shown in FIG. 1 is subjected to gas low-temperature sterilization treatment similar to the endoscope 10A according to the second embodiment.

The endoscope 10B includes a flexible tube part 46 instead of the flexible tube part 36 of the endoscope 10A according to the second embodiment. As shown in FIG. 2, the flexible tube part 46 includes a lubricant layer 47 (a lubricant, an applied layer) instead of the lubricant layer 37 according to the second embodiment.

Hereinafter, a difference between the first modification example and the second embodiment will be focused and described below.

As schematically shown in FIG. 6, the lubricant layer 47 includes an anti-friction material layer 37A and an ion exchanger layer 37B. The anti-friction material layer 37A and the ion exchanger layer 37B are stacked in this order on a surface 30a of an adherend 30. That is, the lubricant layer 47 of this modification example is an example where the stacking order of the ion exchanger layer 37B and the anti-friction material layer 37A of the lubricant layer 37 according to the second embodiment is reversed.

The lubricant layer 47 is manufactured in the same manner as that according to the second embodiment except that the application order of the second material M2 to be applied and the first material M1 to be applied is reversed in the second embodiment.

The lubricant layer 47 of the endoscope 10B of this modification example includes the anti-friction material layer 37A and the ion exchanger layer 37B that are the same as those according to the second embodiment.

As a result, chemical attack on the anti-friction material 17A, which is caused by reactive components permeating the lubricant layer 47 in gas low-temperature sterilization, is suppressed by the action of the ion exchanger 17B.

As a result, according to the lubricant layer 47 of this modification example and the endoscope 10B including the lubricant layer 47, resistance to gas low-temperature sterilization is improved.

However, the ion exchanger layer 37B covers the anti-friction material layer 37A in the form of a layer in this modification example. As a result, reactive components permeating the lubricant layer 47 are likely to be trapped by the ion exchanger layer 37B before reaching the anti-friction material layer 37A. For this reason, chemical attack on the anti-friction material layer 37A is suppressed as compared to the second embodiment. The sterilization resistance of the anti-friction material layer 37A is further improved.

In this modification example, the anti-friction material layer 37A is in a state where the anti-friction material layer 37A is in contact with a sliding counterpart member through the ion exchanger layer 37B. As a result, chemical attack on the sliding counterpart member is also suppressed in a range covered with the ion exchanger layer 37B.

During the use of the endoscope 10B, the sliding counterpart member is in contact with the ion exchanger layer 37B. The ion exchanger layer 37B has not much friction reduction action. However, the layered anti-friction material layer 37A is interposed between the ion exchanger layer 37B and the adherend 30. As a result, the ion exchanger layer 37B and the surface 30a of the adherend 30 are smoothly moved relative to each other due to the shear deformation of the anti-friction material layer 37A.

According to this modification example, sliding friction is further reduced during the use of the endoscope 10B as described above as in the second embodiment.

### SECOND MODIFICATION EXAMPLE

Next, a second modification example according to the second embodiment will be described.

FIG. 7 is a schematic cross-sectional view showing an example of the layer structure of a lubricant for a medical device of a second modification. example according to the second embodiment of the invention.

An endoscope 103 (medical device) of this modification example shown. in FIG. 1 is subjected to gas low-temperature sterilization treatment like the endoscope 10A according to the second embodiment.

The endoscope 10C includes a flexible tube part 56 instead of the flexible tube part 36 of the endoscope 10A according to the second embodiment. As shown in FIG. 2, the flexible tube part 56 includes a lubricant layer 57 (a lubricant, an applied layer) instead of the lubricant layer 37 according to the second embodiment.

A difference between the second modification example and the second embodiment will be mainly described below.

As schematically shown in FIG. 7, the lubricant layer 57 includes an ion exchanger layer 37B, an anti-friction material layer 37A, and an ion exchanger layer 37B. The ion exchanger layer 37B, the anti-friction material layer 37A, and the ion exchanger layer 37B are stacked in this order on a surface 30a of an adherend 30. That is, the lubricant layer 57 of this modification example is an example where the ion exchanger layer 37B is further stacked on the lubricant layer 37 according to the second embodiment. However, the thickness of each of the ion exchanger layer 37B, the anti-friction material layer 37A, and the ion exchanger layer 37B of the lubricant layer 57 may be different from that according to the second embodiment.

It is more preferable that the percentage content of the ion exchanger 17B included in the respective ion exchanger layers 37B of the lubricant layer 57 is in the range of 0.1% by mass to 70% by mass as a whole.

The lubricant layer 57 is manufactured in the same manner as that according to the second embodiment.

The lubricant layer 57 of the endoscope 10C of this modification example includes the anti-friction material layer 37A and the ion exchanger layer 37B that are the same as those according to the second embodiment.

As a result, chemical attack on the anti-friction material 17A, which is caused by reactive components permeating the lubricant layer 57 in gas low-temperature sterilization, is suppressed by the action of the ion exchanger 17B.

According to the lubricant layer 57 of this modification example and the endoscope 10C including the lubricant layer 57, resistance to gas low-temperature sterilization is improved as described above.

However, the. anti-friction material layer 37A is interposed between the ion exchanger layers 37B in this modification example. As a result, as in the first modification example, reactive components permeating the lubricant layer 57 are likely to be trapped by the ion exchanger layer 37B serving as the outermost layer before reaching the anti-friction material layer 37A. In addition, as in the second embodiment, chemical attack on the adherend 30 is suppressed by the ion exchanger layer 37B stacked on the surface 30a of the adherend 30.

Friction reduction action during the use of the endoscope 10C is good as in the first modification example.

Examples of cases where the medical devices using the lubricants of the respective embodiments and the respective modification examples are medical endoscopes have been described in the description of the respective embodiments and the respective modification examples. However, as long as the medical device is a medical device to be subjected to gas low-temperature sterilization, the medical device is not limited to an endoscope. Examples of the medical devices using the lubricants of the respective embodiments and the respective modification examples include a treatment tool, an energy device, and the like.

Examples of cases where the anti-friction material layer and the ion exchanger layer of the lubricant layer are formed of two layers or three layers and the anti-friction material layer and the ion exchanger layer are alternately stacked in the thickness direction have been described in the second embodiment and the respective modification examples. However, the numbers of the anti-friction material layers and the ion exchanger layers of the lubricant layer are not limited thereto.

### EXAMPLES

Examples 1 to 5 of the lubricants for a medical device to be subjected to gas low-temperature sterilization of the first and second embodiments will be described together with Comparative Examples 1 and 2.

Table 1 shows the composition and evaluation results of the lubricants for a medical device to be subjected to gas low-temperature sterilization of Examples 1 to 5 and Comparative Examples 1 and 2.

[Table 1]

TABLE 1

| | | | | | Evaluation result | | | |
|---|---|---|---|---|---|---|---|---|
| | | | | | Coefficient of kinetic | | | |
| | Lubricant | | | | friction (measured value) | | | |
| | | | Percentage | | | Alter 200 | | |
| | | | content of | | Before | times of | | |
| | | | second | | sterilization | sterilization | | |
| | First | Second | component | Application | treatment | treatment | Variation | Comprehensive |
| | component | component | (% by mass) | type | (A) | (B) | B − A | evaluation |
| Example 1 | MoS₂ | Ion | 70 | Mixing | 0.180 | 0.195 | 0.015 | A |
| | | exchanger | | application | | | | |
| Example 2 | MoS₂ | Ion | 75 | Mixing | 0.175 | 0.206 | 0.031 | B |
| | | exchanger | | application | | | | |
| Example 3 | MoS₂ | Ion | 0.1 | Mixing | 0.100 | 0.189 | 0.089 | A |
| | | exchanger | | application | | | | |
| Example 4 | MoS₂ | Ion | 0.05 | Mixing | 0.139 | 0.210 | 0.071 | B |
| | | exchanger | | application | | | | |
| Example 5 | MoS₂ | Ion | 70 | Two-layer | 0.110 | 0.182 | 0.072 | A |
| | | exchanger | | division | | | | |
| | | | | application | | | | |
| Comparative | MoS₂ | none | — | Single-layer | 0.117 | 0.262 | 0.145 | C |
| Example 1 | | | | application | | | | |
| Comparative | MoS₂ | Pt | 10 | Mixing | 0.155 | 0.250 | 0.095 | C |
| Example 2 | | | | application | | | | |

### Example 1

Example 1 is Example of the lubricant layer 17 of the first embodiment. As shown in Table 1, a lubricant of Example 1 includes molybdenum disulfide as a first component and includes an ion exchanger as a second component. The molybdenum disulfide is an example of an anti-friction material.

The molybdenum disulfide is prepared as powder having an average particle size of 1.0 μm.

IXE (registered trademark)-6107 (trade name; manufactured by Toagosei Co., Ltd.) is used as the ion exchanger. IXE (registered trademark)-6107 (trade name; manufactured by Toagosei Co., Ltd.) is an inorganic amphoteric ion exchanger.

The molybdenum disulfide and the ion exchanger are mixed with each other in order to prepare a material to be applied. A mixing ratio of the molybdenum disulfide to the ion exchanger is set to 3:7 by mass. Accordingly, the material to be applied is prepared.

A planar silicone base material haying a size of 100 mm×100 mm is used as an adherend used to form an evaluation sample. A silicone rubber sheet (manufactured by AS ONE Corporation) is used as the silicone base material.

The material to be applied is applied to the silicone base material by a dry method (mixing application). The thickness of an applied layer is set to 20 μm. Accordingly, an evaluation sample of Example 1 is formed. In the lubricant of this evaluation sample, the percentage content of the ion exchanger is set to 70% by mass.

### Examples 2 to 4

An evaluation sample of Example 2 is formed in the same manner as that of Example 1 except that the percentage content of the ion exchanger is set to 75% by mass.

An evaluation sample of Example 3 is formed in the same manner as that of Example 1 except that the percentage content of the ion exchanger is set to 0.1% by mass.

An evaluation sample of Example 4 is formed in the same manner as that of Example 1 except that the percentage content of the ion exchanger is set to 0.05% by mass.

### Example 5

Example 5 is Example of the lubricant layer 37 according to the second embodiment.

A lubricant of Example 5 includes a first component and a second component that are the same as those of Example 1.

A first material M1 to be applied formed of an ion exchanger and a second material M2 to be applied made of molybdenum disulfide are prepared in order to manufacture an evaluation sample of Example 5

The first material M1 to be applied is applied to the same silicone base material as that of Example 1 by a dry method. The thickness of an applied layer is set to 10 μm. Accordingly, an ion exchanger layer 37B is formed. The second material M2 to be applied is applied to the ion exchanger layer 37B by a dry method. The thickness of an applied layer is set to 10 μm. Accordingly, the evaluation sample of Example 5 is formed. In the lubricant of this evaluation sample, the percentage content of the ion exchanger is set to 70% by mass as in Example 1.

### Comparative Examples 1 and 2

An evaluation sample of Comparative Example 1 is different from that of Example 1 in that only molybdenum disulfide is used as a lubricant.

Molybdenum disulfide is applied to the same silicone base material as that of Example 1 by a dry method, so that the evaluation sample of Comparative Example 1 is manufactured (single-layer application). The thickness of an applied layer is set to 20 μm.

In an evaluation sample of Comparative Example 2, platinum (Pt) is used instead of the ion exchanger of Example 1. The percentage content of platinum in the lubricant is set to 10% by mass.

The lubricant of Comparative Example 2 is applied to a silicone base material by the same mixing application as Example 1.

### Evaluation

The evaluation sample of each Example and the evaluation sample of each Comparative Example are subjected to gas low-temperature sterilization 200 times. The gas low-temperature sterilization is performed by a hydrogen peroxide low-temperature plasma sterilization method using STERRAD (registered trademark) NX (registered trademark) (trade name; manufactured by Johnson & Johnson K.K.).

The coefficient of kinetic friction of the lubricant of the evaluation sample is measured before sterilization is performed and after sterilization is performed 200 times. A surface property tester TRIBIGEAR (registered trademark) TYPE: 14FW (trade name; manufactured by Shinto Scientific Co., Ltd.) is used for the measurement of the coefficient of kinetic friction. A stainless steel plate having a thickness of 1 mm and a width of 25 mm is used as a counterpart member. Test conditions include a speed of 1000 mm/min, a stroke of 15 mm, 500 times of reciprocation, and an applied load of 500 gf (4.9 N).

A comprehensive evaluation is made as three levels of “very good” (“A” in Table 1), “good” (“B” in Table 1), and “no good” (“C” in Table 1).

A comprehensive evaluation in a case where a coefficient of kinetic friction after sterilization treatment is 0.195 or less is defined as “very good”.

A comprehensive evaluation in a case where a coefficient of kinetic friction after sterilization treatment is higher than 0.195 and lower than 0.220 is defined as “good”.

Comprehensive evaluation in a case where a coefficient of kinetic friction after sterilization treatment is higher than 0.220 is defined as “no good”.

### Evaluation Result

As shown in Table 1, the coefficients of kinetic friction of Examples 1 to 5 before sterilization treatment are 0.180, 0.175, 0.100, 0.139, and 0.110, respectively. The coefficients of kinetic friction of Examples 1 to 5 after 200 times of sterilization treatment are 0.195, 0.206, 0.189, 0.210, and 0.182, respectively.

The coefficients of kinetic friction of Comparative Examples 1 and 2 before sterilization treatment are 0.117 and 0.155, respectively. The coefficients of kinetic friction of Comparative Examples 1 and 2 after 200 times of sterilization treatment are 0.262 and 0.250, respectively.

Since the coefficients of kinetic friction of all Examples and Comparative Examples are increased after sterilization treatment, it is regarded that the friction characteristics of a lubricant deteriorate due to sterilization treatment. It is thought that the degree of deterioration of friction characteristics corresponds to the amount of reacting molybdenum disulfide. Accordingly, it is thought that sulfurous acid, sulfuric acid, and the like are generated according to the degree of deterioration of friction characteristics.

Since the coefficients of kinetic friction of Examples 1, 3, and 5 after sterilization treatment are 0.195 or less, Examples 1, 3, and 5 are evaluated as “very good”.

Since the coefficients of kinetic friction of Examples 2 and 4 after sterilization treatment are higher than 0.195 and lower than 0.220, Examples 2 and 4 are evaluated as “good”.

In contrast, both the comprehensive evaluations of Comparative Examples 1 and 2 are “no good”.

In a case where mixing application is used and the percentage content of the ion exchanger is high as in Examples 1 and 2, a variation in the coefficient of kinetic friction after sterilization treatment is small. It is thought that the reason for this is that reactive components permeating from the outside of the evaluation sample can be efficiently trapped since a lot of ion exchanger is distributed on the surface of the lubricant layer.

In a case where mixing application is used and the percentage content of the ion exchanger is low as in Examples 3 and 4, a variation in the coefficient of kinetic friction after sterilization treatment is larger than those of Examples 1 and 2. It is thought that the reason for this is that the amount of ion exchanger on the surface of the lubricant layer is reduced as compared to those of Examples 1 and 2. However, since the amount of ion exchanger in the lubricant layer is small, the coefficients of kinetic friction of Examples 3 and. 4 before sterilization treatment are smaller than those of Examples 1 and 2. As a result, the coefficients of kinetic friction of Examples 3 and 4 after sterilization treatment are also in ranges that are evaluated as “very good” and “good”.

Since the percentage content of the ion exchanger is high but the ion exchanger is covered with the anti-friction material in Example 5, a variation in the coefficient of kinetic friction of Example 5 is substantially equal to that of Example 4. However, the ion exchanger is not included in the anti-friction material layer in Example 5. As a result, it is thought that a coefficient of kinetic friction, which is substantially equal to a coefficient of kinetic friction in a case where the content of the ion exchanger is small as in Example 3, is obtained in Example 5. As a result, it is thought that the comprehensive evaluation of Example 5 is “very good”.

In contrast, since the ion exchanger is not used in Comparative Example 1, a variation in a coefficient of kinetic friction is significantly increased. As a result, it is thought that the comprehensive evaluation of Comparative Example 1 is “no good”.

Since platinum thought to have a catalytic action on oxygenated water is included in Comparative Example 2, a variation in the coefficient of kinetic friction of Comparative Example 2 is smaller than that of Comparative Example 1. However, in a case where Comparative Example 2 is compared with Examples 1 to 5, a variation in the coefficient of kinetic friction of Comparative Example 2 is larger than those of Examples 1 to 5. Further, since platinum particles are included in Comparative Example 2, a coefficient of kinetic friction before sterilization is not much lowered. For this reason, a coefficient of kinetic friction of Comparative Example 2 after sterilization treatment is high. As a result, the comprehensive evaluation of Comparative Example 2 is “no good”.

The respective preferred embodiments, the respective modification examples, and the respective examples of the invention have been described above, but the invention is not limited to the respective embodiments, the respective modification examples, and the respective examples. Elements can be added, omitted, and substituted, and the other modifications may be applied without departing from the scope of the invention.

Further, the invention is not limited by the above description. and is limited by only accompanying claims.

For example, a layer in which the anti-friction material 17A and the ion exchanger 17B are mixed with each other like the lubricant layer 17 of the first embodiment and at least one of the anti-friction material layer 37A and the ion exchanger layer 37B according to the second embodiment may be stacked to form a lubricant layer.

For example, a structure where an anti-friction material 17A patterned in the shape of dots or the like and an ion exchanger 17B patterned in the shape of dots or the like are independently distributed with a gap therebetween on an adherend may be used in the first embodiment. Such a structure is an example of a special case where particles of the anti-friction material 17A or the aggregate thereof and particles of the ion exchanger 17B or the aggregate thereof are away from each other.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. A lubricant for a medical device to be subjected to gas low-temperature sterilization, the lubricant comprising:
an anti-friction material; and
an ion exchanger.

2. The lubricant for a medical device to be subjected to gas low-temperature sterilization according to claim 1,
wherein the ion exchanger contains an inorganic substance that discharges at least one of a hydroxide ion and a proton.

3. The lubricant for a medical device to be subjected to gas low-temperature sterilization according to claim 1,
wherein a content of the ion exchanger is in a range of 0.1% by mass to 70% by mass.

4. The lubricant for a medical device to be subjected to gas low-temperature sterilization according to claim 1,
wherein the ion exchanger and the anti-friction material are mixed with each other.

5. The lubricant for a medical device to be subjected to gas low-temperature sterilization according to claim 1,
wherein the anti-friction material contains molybdenum disulfide.

6. A medical device to be subjected to gas low-temperature sterilization comprising:
the lubricant according to claim 1.

7. The medical device to be subjected to gas low-temperature sterilization. according to claim 6,
wherein the lubricant is provided in a form of a layer on a surface of an adherend.

8. The medical device to be subjected to gas low-temperature sterilization according to claim 7,
wherein the lubricant includes an anti-friction material layer that includes the anti-friction material as a main component, and an ion exchanger layer that includes the ion exchanger as a main component, and
the anti-friction material layer and the ion exchanger layer are alternately arranged in a thickness direction.

9. The medical device to be subjected to gas low-temperature sterilization according to claim 6,
wherein the medical device is an endoscope.

10. The medical device to be subjected to gas low-temperature sterilization according to claim 9,
wherein the endoscope includes a flexible tube and an insertion member that is inserted into the flexible tube, and
the lubricant is provided between an inner peripheral surface of the flexible tube and an outer peripheral surface of the insertion member.

11. A method of manufacturing a medical device to be subjected to gas low-temperature sterilization, the method comprising:
applying a lubricant including an anti-friction material and an ion exchanger to at least part of a device body of a medical device to be subjected to gas low-temperature sterilization.

12. The method of manufacturing a medical device to be subjected to gas low-temperature sterilization according to claim 11,
wherein the process of applying the lubricant comprises preparing a material to be applied in which at least the anti-friction material and the ion exchanger are mixed with each other, and applying the material to be applied to the device body.

13. The method of manufacturing a medical device to be subjected to gas low-temperature sterilization according to claim 12,
wherein the process of applying the lubricant comprises forming two or more applied layers including a component of the lubricant on the device body, and
wherein the applied layers are formed by alternately arranging a lubricant layer including the anti-friction material as a main component and an ion exchanger layer including the ion exchanger as a main component.
